(19) United States
(12) Patent Application Publication
(10) Pub. No.: US 2006/0293298 A1
(43) Pub. Date: Dec. 28, 2006

(54) Compounds

(76) Inventors: Mark James Bamford, Harlow (GB); David Kenneth Dean, Harlow (GB); Ashley Paul Hancock, Stevenage (GB); David Matthew Wilson, Harlow (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/246,480

(22) Filed: Oct. 7, 2005

Related U.S. Application Data
(63) Continuation-in-part of application No. 10/551,985, filed on Oct. 4, 2005.

(30) Foreign Application Priority Data
Apr. 10, 2003 (GB) 0308333.4
May 25, 2005 (GB) 0510731.3

(51) Int. Cl. A61K 31/4545; C07D 401/14

(52) U.S. Cl. 514/210.18; 546/187

(57) ABSTRACT

The present invention relates to 1-{[4-(1-Azetidinylcarbonyl)phenyl]carbonyl}-4-(4-{[1-(1-methylethyl)-4-piperidinyl]oxy}phenyl)piperidine 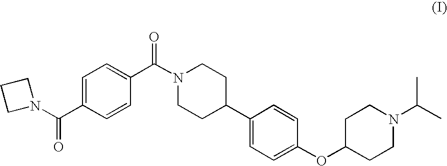 and derivatives thereof, and to compositions, processes for its preparation and its uses in therapy.

### CROSS-REFERENCE TO RELATED APPLICATIONS

This application is a continuation-in-part of copending U.S. patent application No. (to be provided) filed 4 Oct. 2005, which is the US national phase of PCT International Application No. PCT/EP2004/003985 filed on 8 Apr. 2004, which designates the United States, and which claims priority under 35 USC 119 on British Application No. GB0308333.4, which was filed on 10 Apr. 2003. This application also claims priority on British Application No. GB 0510731.3, which was filed on 25 May 2005. The entire contents of all the above applications are incorporated herein by reference.

### FIELD OF THE INVENTION

The present invention relates to compounds, processes for their preparation, pharmaceutical compositions containing them and to their use in the treatment of various disorders, in particular inflammatory and/or allergic disorders of the respiratory tract.

### BACKGROUND OF THE INVENTION

Allergic rhinitis, pulmonary inflammation and congestion are medical conditions that are often associated with other conditions such as asthma, chronic obstructive pulmonary disease (COPD), seasonal allergic rhinitis and perennial allergic rhinitis. In general these conditions are mediated, at least in part, by inflammation associated with the release of histamine from various cells, in particular mast cells.

Allergic rhinitis, also known as ‘hay fever’ affects a large proportion of the population worldwide. There are two types of allergic rhinitis, seasonal and perennial. The clinical symptoms of seasonal allergic rhinitis, typically include nasal itching and irritation, sneezing and watery rhinorrhea which is often accompanied by nasal congestion. The clinical symptoms of perennial allergic rhinitis are similar except that nasal blockage may be more pronounced. Either type of allergic rhinitis may also cause other symptoms such as itching of the throat and/or eyes, epiphora and oedema around the eyes. The symptoms of allergic rhinitis may vary in intensity from the nuisance level to debilitating.

Allergic rhinitis and other allergic conditions are associated with the release of histamine from various cell types but particularly mast cells. The physiological effects of histamine are classically mediated by three receptor subtypes, termed H1, H2 and H3. H1 receptors are widely distributed throughout the CNS and periphery, and are involved in wakefulness and acute inflammation. H2 receptors mediate gastric acid secretion in response to histamine. H3 receptors are present on the nerve endings in both the CNS and periphery and mediate inhibition of neurotransmitter release [Hill et al, Pharmacol. Rev. 49:253-278 (1997)]. Recently a fourth member of the histamine receptor family has been identified, termed the H4 receptor [Hough, Mol. Pharmacol. 59: 415-419, (2001)]. Whilst the distribution of the H4 receptor appears to be restricted to cells of the immune and inflammatory systems, a physiological role for this receptor remains to be identified.

The activation of H1 receptors in blood vessels and nerve endings are responsible for many of the symptoms of allergic rhinitis, which include itching, sneezing, and the production of watery rhinorrhea. Antihistamine compounds, i.e. drugs which are selective H1 receptor antagonists such as chlorphenyramine and cetirizine, are effective in treating the itching, sneezing and rhinorrhea associated with allergic rhinitis, but are not effective against the nasal congestion symptoms [Aaronson, Ann. Allergy, 67:541-547, (1991)].

Histamine H3 receptors are expressed widely on both CNS and peripheral nerve endings and mediate the inhibition of neurotransmitter release. In vitro electrical stimulation of peripheral sympathetic nerves in isolated human saphenous vein results in an increase in noradrenaline release and smooth muscle contraction, which can be inhibited by histamine H3 receptor agonists [Molderings et al, Naunyn-Schmiedeberg's Arch. Pharmacol., 346: 46-50, (1992); Valentine et al,. Eur. J. Pharmacol., 366: 73-78, (1999)]. H3 receptor agonists also inhibit the effect of sympathetic nerve activation on vascular tone in porcine nasal mucosa [Varty & Hey. Eur. J. Pharmacol., 452:339-345, (2002)]. In vivo, H3 receptor agonists inhibit the decrease in nasal airway resistance produced by sympathetic nerve activation [Hey et al, Arzneim-Forsch Drug Res., 48:881-888 (1998)]. Activation of histamine H3 receptors in human nasal mucosa inhibits sympathetic vasoconstriction [Varty et al. Eur. J. Pharmacol., 484:83-89, (2004)]. Furthermore, H3 receptor antagonists in combination with histamine H1 receptor antagonists have been shown to reverse the effects of mast cell activation on nasal airway resistance and nasal cavity volume, an index of nasal congestion [Mcleod et al, Am. J. Rhinol.,13:391-399, (1999)], and further evidence for the contribution of H3 receptors to histamine-induced nasal blockage is provided by histamine nasal challenge studies performed on normal human subjects [Taylor-Clark et al. British J. Pharmacol., 1-8 (2005)].

### SUMMARY OF THE INVENTION

The present invention relates to a compound (or derivative thereof) that is a histamine H3 antagonist and/or inverse agonist. This compound (or derivative thereof) may be useful in the treatment of various disorders in particular inflammatory and/or allergic disorders, such as inflammatory and/or allergic disorders of the respiratory tract, for example allergic rhinitis, that are associated with the release of histamine from cells such as mast cells. Further, the compound of the invention (or derivative thereof may show an improved profile over known H3 antagonists/inverse agonists in that it may possess one or more of the following properties:
(i) potent H3 antagonist/inverse agonist activity with a pKi of greater than about 9.5;
(ii) selective for the H3 receptor over the H1 receptor;
(iii) low CNS penetration;
(iv) improved bioavailability; and
(v) lower clearance and/or longer half-life in blood.

Compounds having such a profile may be orally effective, and/or capable of once daily administration and/or further may have an improved side effect profile compared with other existing therapies.

Thus the present invention provides, in a first aspect, the compound 1-{[4-(1-azetidinylcarbonyl)phenyl]carbonyl}-4-(4-{[1-(1-methylethyl)-4-piperidinyl]oxy}phenyl)piperidine 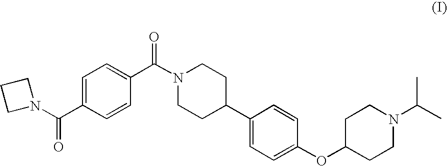
or a derivative thereof, such as a pharmaceutically acceptable derivative e.g. a salt.

It is to be understood that the present invention covers the compound of formula (I) as the free base and as a derivative thereof e.g. a salt, such as a pharmaceutically acceptable derivative e.g. a pharmaceutically acceptable salt.

As used herein, the term “pharmaceutically acceptable derivative”, means any pharmaceutically acceptable salt or solvate of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. Representative pharmaceutically acceptable derivatives are salts and solvates. Exemplary pharmaceutically acceptable derivatives are salts and solvates, particularly salts.

It will be appreciated that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention.

The compounds of the present invention may be in the form of and/or may be administered as a pharmaceutically acceptable salt. Pharmaceutically acceptable salts include acid addition salts. For a review on suitable salts see Berge et al., J. Pharm. Sci., 1977, 66, 1-19.

A pharmaceutically acceptable acid addition salt may be readily prepared by using a desired acid as appropriate. The salt may precipitate from solution and be collected by filtration or may be recovered by evaporation of the solvent. Typically a pharmaceutically acceptable acid addition salt can be formed by reaction of a compound of formula (I) with a suitable inorganic or organic acid (such as hydrobromic, hydrochloric, formic, sulfuric, nitric, phosphoric, succinic, maleic, acetic, fumaric, citric, tartaric, benzoic, p-toluenesulfonic, methanesulfonic or naphthalenesulfonic acid), optionally in a suitable solvent such as an organic solvent, to give the salt which is usually isolated for example by crystallisation and filtration. Thus, a pharmaceutically acceptable acid addition salt of a compound of formula (I) can be for example a hydrobromide, hydrochloride, formate, sulfate, nitrate, phosphate, succinate, maleate, acetate, fumarate, citrate, tartrate, benzoate, p-toluenesulfonate, methanesulfonate or naphthalenesulfonate salt.

Other non-pharmaceutically acceptable salts, eg. oxalates or trifluoroacetates, may be used, for example in the isolation of compounds of the invention, and are included within the scope of this invention. The invention includes within its scope all possible stoichiometric and non-stoichiometric forms of the salts of the compounds of formula (I).

Included within the scope of the invention are all solvates, hydrates, complexes and polymorphic forms of the compound and derivatives e.g. salts of the invention.

The present invention also provides a process for the preparation of a compound of formula (I) or a derivative thereof.

Thus a compound of formula (I) may be prepared by reacting 4-(azetidinyl-1-ylcarbonyl)benzoic acid shown in formula (II) 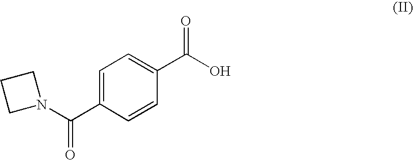
with a compound of formula (III) 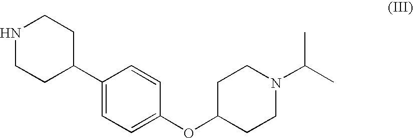
or a derivative thereof such as a salt, for example an acid addition salt.

The reaction may be carried out in the presence of a suitable base such as diisopropylethylamine and a suitable coupling agent such as O-(benzotriazol-1-yl)-N,N,N′,N′-tetramethyluronium tetrafluoroborate (TBTU), in an appropriate solvent such as dichloromethane.

Compounds of formula (III) may be prepared in accordance with the following or similar reaction scheme:

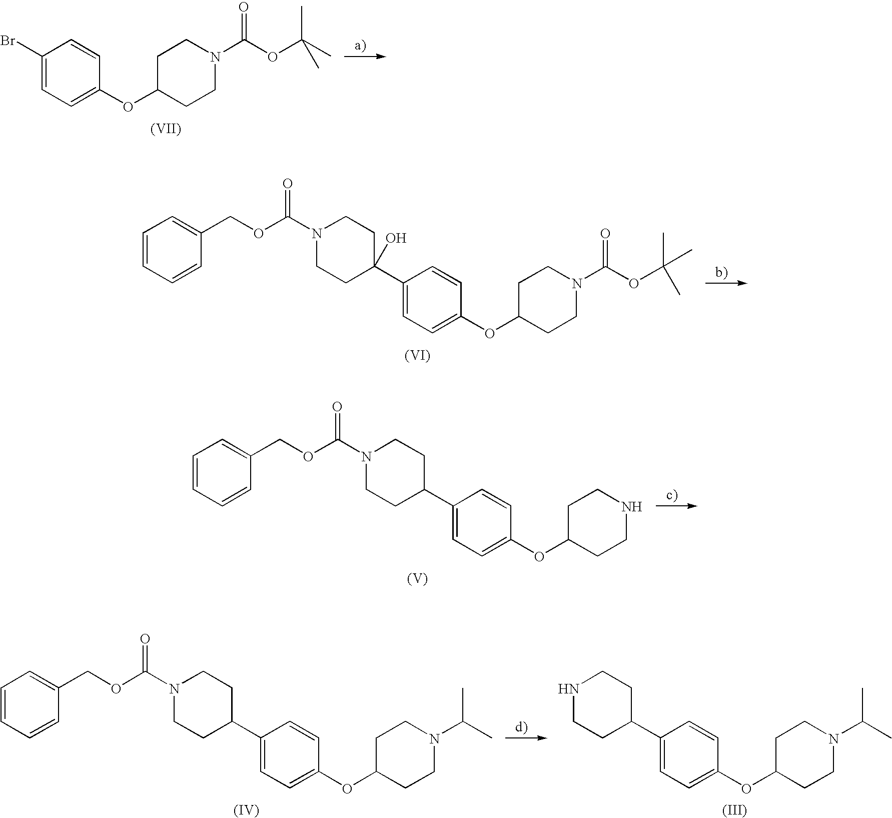

Reagents and Conditions: a) n-butyl lithium in hexane, phenylmethyl 4-oxo-1-piperidinecarboxylate, tetrahydrofuran; b) trifluoroacetic acid, triethylsilane, dichloromethane, −78° C.; c) acetone, sodium triacetoxyborohydride; d) H₂, 10% palladium on carbon, ethanol.

A compound of formula (VI) is known (see Description 5 in WO0489373). A compound of formula (VII) is known (see Example 22(a) in WO0069819).

Examples of protecting groups that may be employed in the synthetic routes described and the means for their removal can be found in T. W. Greene ‘Protective Groups in Organic Synthesis’ (3rd edition, J. Wiley and Sons, 1999). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrogen chloride in dioxan or trifluoroacetic acid in dichloromethane) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

Examples of disease states in which a compound of formula (I), or a pharmaceutically acceptable derivative thereof may have potentially beneficial anti-inflammatory and/or anti-allergic effects include diseases of the respiratory tract such as bronchitis (including chronic bronchitis), asthma (including allergen-induced asthmatic reactions), chronic obstructive pulmonary disease (COPD), cystic fibrosis, sinusitis and allergic rhinitis (seasonal and perennial). Other disease states include diseases of the gastrointestinal tract such as intestinal inflammatory diseases including inflammatory bowel disease (e.g. Crohn's disease or ulcerative colitis) and intestinal inflammatory diseases secondary to radiation exposure or allergen exposure.

Furthermore, compound of the invention may be used to treat nephritis, skin diseases such as psoriasis, eczema, allergic dermatitis and hypersensitivity reactions.

Compound of the invention may also be of use in the treatment of nasal polyposis, conjunctivitis or pruritis.

Further diseases include inflammatory diseases of the gastrointestinal tract such as inflammatory bowel disease.

A disease of particular interest is allergic rhinitis.

Compounds that are antagonists and/or inverse agonists of the H3 receptor may also be of use in other diseases in which activation of the H3 receptor may be implicated. Such diseases may include non-allergic rhinitis.

It will be appreciated by those skilled in the art that references herein to treatment or therapy extend to prophylaxis as well as the treatment of established conditions.

As mentioned above, compounds of formula (I) are useful as therapeutic agents. There is thus provided, as a further aspect of the invention, a compound of formula (I) or a pharmaceutically acceptable derivative thereof for use in therapy.

According to another aspect of the invention, there is provided the use of a compound of formula (I) or a pharmaceutically acceptable derivative thereof for the manufacture of a medicament for the treatment of any of the above diseases.

In a further aspect there is provided a method for the treatment of any of the above diseases, in a human or animal subject in need thereof, which method comprises administering an effective amount of a compound of formula (I) or a pharmaceutically acceptable derivative thereof such as a pharmaceutically acceptable salt or solvate.

When used in therapy, the compounds of formula (I) are usually formulated in a suitable pharmaceutical composition. Such compositions can be prepared using standard procedures.

Thus, the present invention further provides a pharmaceutical composition which comprises a compound of formula (I) or a pharmaceutically acceptable derivative thereof optionally with one or more pharmaceutically acceptable carriers and/or excipients.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colorants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. A surfactant or wetting agent may be included in the composition to facilitate uniform distribution of the compound.

The composition may contain from about 0.1% to 99% by weight, such as from about 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be about 0.05 to 1000 mg, more suitably about 1.0 to 200 mg, and such unit doses may be administered more than once a day, for example two or three a day. Such therapy may extend for a number of weeks or months. In one embodiment compounds and pharmaceutical composition according to the invention are suitable for oral administration and/or are capable of once daily administration.

The compound and pharmaceutical compositions according to the invention may be used in combination with or include one or more other therapeutic agents, for example selected from anti-inflammatory agents, anticholinergic agents (particularly an M₁/M₂/M₃ receptor antagonist), β₂-adrenoreceptor agonists, antiinfective agents such as antibiotics or antivirals, or other antihistamines. The invention thus provides, in a further aspect, a combination comprising a compound of formula (I) or a pharmaceutically acceptable derivative thereof such as a salt or solvate together with one or more other therapeutically active agents, for example selected from an anti-inflammatory agent such as a corticosteroid or an NSAID, an anticholinergic agent, a β₂-adrenoreceptor agonist, an antiinfective agent such as an antibiotic or an antiviral, or another antihistamine. Combinations comprising a compound of formula (I) or a pharmaceutically acceptable salt, solvate or derivative thereof together with a corticosteroid and/or another antihistamine form yet another aspect of the present invention. The combinations of the invention may optionally include one or more pharmaceutically acceptable carriers and/or excipients as desired.

Of interest is a combination comprising a compound of formula (I) or a pharmaceutically acceptable derivative thereof together with an H1 antagonist. Suitable H1 antagonists include without limitation astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine and triprolidine, particularly cetirizine, levocetirizine, efletirizine and fexofenadine.

Examples of β₂-adrenoreceptor agonists include salmeterol (which may be a racemate or a single enantiomer, such as the R-enantiomer), salbutamol, formoterol, salmefamol, fenoterol or terbutaline and salts thereof, for example the xinafoate salt of salmeterol, the sulphate salt or free base of salbutamol or the fumarate salt of formoterol. Long-acting β₂-adrenoreceptor agonists such as salmeterol or formoterol may be preferred.

Exemplary long acting β₂-adrenoreceptor agonists include those described in WO02/66422A, WO02/270490, WO02/076933, WO03/024439 and WO03/072539.

Anti-inflammatory agents include corticosteroids. Corticosteroids which may be used in combination with the compound of the invention are those oral and inhaled corticosteroids and their pro-drugs which have anti-inflammatory activity. Examples include methyl prednisolone, prednisolone, dexamethasone, fluticasone propionate, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-17α-(1-methylcylopropylcarbonyl)oxy-3-oxo-androsta-1,4-diene-17β-carbothioic acid S-fluoromethyl ester, 6α,9α-difluoro-11β-hydroxy-16α-methyl-3-oxo -17α-(2,2,3,3-tetramethylcyclopropylcarbonyl)oxy-androsta-1,4-diene-17β-carboxylic acid cyanomethyl ester, beclomethasone esters (such as the 17-propionate ester or the 17,21-dipropionate ester), budesonide, flunisolide, mometasone esters (such as the furoate ester), triamcinolone acetonide, rofleponide, ciclesonide, (16α,17-[[(R)-cyclohexylmethylene]bis(oxy)]-11β,21-dihydroxy-pregna-1,4-diene-3,20-dione), butixocort propionate, RPR-106541, and ST-126. Corticosteroids that may be preferred include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, particularly 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Non-steroidal compounds having glucocorticoid agonism that may possess selectivity for transrepression over transactivation and that may be useful in combination therapy include those covered in the following patents: WO03/082827, WO01/10143, WO98/54159, WO04/005229, WO04/009016, WO04/009017, WO04/018429, WO03/104195, WO03/082787, WO03/082280, WO03/059899, WO03/101932, WO02/02565, WO01/16128, WO/0066590, WO03/086294, WO04/026248, WO03/061651, WO03/08277.

Anti-inflammatory agents include non-steroidal anti-inflammatory drugs (NSAID's).

NSAID's include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis (eg. montelukast), iNOS (inducible nitric oxide synthase) inhibitors, tryptase and elastase inhibitors, beta-2 integrin antagonists and adenosine receptor agonists or antagonists (e.g. adenosine 2a agonists), cytokine antagonists (e.g. chemokine antagonists, such as a CCR3 antagonist) or inhibitors of cytokine synthesis, or 5-lipoxygenase inhibitors. iNOS inhibitors include those disclosed in WO93/13055, WO98/30537, WO02/50021, WO95/34534 and WO99/62875. CCR3 inhibitors include those disclosed in WO02/26722.

PDE4-specific inhibitors that may be useful in this aspect of the invention include any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds which may be of interest include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 03 Sep., 1996.

Other PDE4 inhibitors include AWD-12-281 from Elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; and arofylline under development by Almirall-Prodesfarma; VM554/UM565 from Vernalis; or T-440 (Tanabe Seiyaku; Fuji, K. et al. J Pharmacol Exp Ther,1998, 284(1): 162), and T2585.

Further compounds which may be of interest are disclosed in the published international patent application WO04/024728 (Glaxo Group Ltd), PCT/EP2003/014867 (Glaxo Group Ltd) and PCT/EP2004/005494 (Glaxo Group Ltd).

Anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (for example, CAS 28797-61-7), darifenacin (for example, CAS 133099-04-4, or CAS 133099-07-7 for the hydrobromide sold under the name Enablex), oxybutynin (for example, CAS 5633-20-5, sold under the name Ditropan), terodiline (for example, CAS 15793-40-5), tolterodine (for example, CAS 124937-51-5, or CAS 124937-52-6 for the tartrate, sold under the name Detrol), otilonium (for example, as the bromide, CAS 26095-59-0, sold under the name Spasmomen), trospium chloride (for example, CAS 10405-02-4) and solifenacin (for example, CAS 242478-37-1, or CAS 242478-38-2, or the succinate also known as YM-905 and sold under the name Vesicare).

Other anticholinergic agents include compounds of formula (XXI), which are disclosed in U.S. patent application 60/487981: 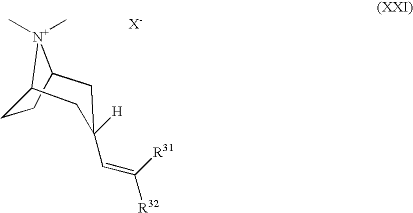
in which the preferred orientation of the alkyl chain attached to the tropane ring is endo;

R³¹ and R³² are, independently, selected from the group consisting of straight or branched chain lower alkyl groups having preferably from 1 to 6 carbon atoms, cycloalkyl groups having from 5 to 6 carbon atoms, cycloalkyl-alkyl having 6 to 10 carbon atoms, 2-thienyl, 2-pyridyl, phenyl, phenyl substituted with an alkyl group having not in excess of 4 carbon atoms and phenyl substituted with an alkoxy group having not in excess of 4 carbon atoms;

X⁻ represents an anion associated with the positive charge of the N atom. X⁻ may be but is not limited to chloride, bromide, iodide, sulfate, benzene sulfonate, and toluene sulfonate, including, for example:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methylbenzenesulfonate;
(3-endo)-8,8-dimethyl-3-[2-phenyl-2-(2-thienyl)ethenyl]-8-azoniabicyclo[3.2.1]octane bromide; and/or
(3-endo)-8,8-dimethyl-3-[2-phenyl-2-(2-pyridinyl)ethenyl]-8-azoniabicyclo[3.2.1]octane bromide.

Further anticholinergic agents include compounds of formula (XXII) or (XXIII), which are disclosed in U.S. patent application 60/511009: 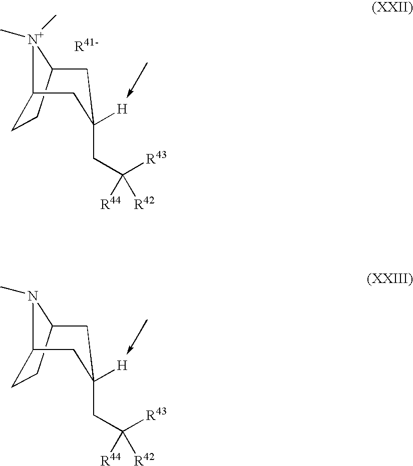
wherein:

the H atom indicated is in the exo position;

R⁴¹⁻ represents an anion associated with the positive charge of the N atom. R1⁻ may be but is not limited to chloride, bromide, iodide, sulfate, benzene sulfonate and toluene sulfonate;

R⁴² and R⁴³ are independently selected from the group consisting of straight or branched chain lower alkyl groups (having preferably from 1 to 6 carbon atoms), cycloalkyl groups (having from 5 to 6 carbon atoms), cycloalkyl-alkyl (having 6 to 10 carbon atoms), heterocycloalkyl (having 5 to 6 carbon atoms) and N or O as the heteroatom, heterocycloalkyl-alkyl (having 6 to 10 carbon atoms) and N or O as the heteroatom, aryl, optionally substituted aryl, heteroaryl, and optionally substituted heteroaryl;

R⁴⁴ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl, —OR⁴⁵, —CH₂OR⁴⁵, —CH₂OH, —CN, —CF₃, —CH₂O (CO)R⁴⁶, —CO₂R⁴⁷, —CH₂NH₂, —CH₂N(R⁴⁷)SO₂R⁴⁵, —SO₂N(R⁴⁷)(R⁴⁸), —CON(R⁴⁷)(R⁴⁸), —CH₂N(R⁴⁸)CO(R⁴⁶), —CH₂N(R⁴⁸)SO₂(R⁴⁶), —CH₂N(R⁴⁸)CO₂(R⁴⁵), CH₂N(R⁴⁸)CONH(R⁴⁷);

R⁴⁵ is selected from the group consisting of (C₁-C₆)alkyl, (C₁-C₆)alkyl(C₃-C₁₂)cycloalkyl, (C₁-C₆)alkyl(C₃-C₇)heterocycloalkyl, (C₁-C₆)alkyl-aryl, (C₁-C₆)alkyl-heteroaryl;

R⁴⁶ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl;

R⁴⁷ and R⁴⁸ are, independently, selected from the group consisting of H, (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, (C₁-C₆)alkyl-aryl, and (C₁-C₆)alkyl-heteroaryl, including, for example:

(Endo)-3-(2-methoxy-2,2-di-thiophen-2-yl-ethyl)-8,8-dimethyl-8-azonia-bicyclo[3.2.1]octane iodide;

3-((Endo)-8-methyl-8-aza-bicyclo[3.2.1]oct-3-yl)-2,2-diphenyl-propionitrile;

(Endo)-8-methyl-3-(2,2,2-triphenyl-ethyl)-8-aza-bicyclo[3.2.1]octane;

3-((Endo)-8-methyl-8-aza-bicyclo[3.2.1]oct-3-yl)-2,2-diphenyl-propionamide;
3-((Endo)-8-methyl-8-aza-bicyclo[3.2.1]oct-3-yl)-2,2-diphenyl-propionic acid;
(Endo)-3-(2-cyano-2,2-diphenyl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane bromide;
3-((Endo)-8-methyl-8-aza-bicyclo[3.2.1]oct-3-yl)-2,2-diphenyl-propan-1-ol;
N-Benzyl-3-((endo)-8-methyl-8-aza-bicyclo[3.2.1]oct-3-yl)-2,2-diphenyl-propionamide;
(Endo)-3-(2-carbamoyl-2,2-diphenyl-ethyl)-8,8-dimethyl-8-azonia-bicyclo[3.2.1]octane iodide;
1-Benzyl-3-[3-((endo)-8-methyl-8-aza-bicyclo[3.2.1]oct-3-yl)-2,2-diphenyl-propyl]-urea;
1-Ethyl-3-[3-((endo)-8-methyl-8-aza-bicyclo[3.2.1]oct-3-yl)-2,2-diphenyl-propyl]-urea;
N-[3-((Endo)-8-methyl-8-aza-bicyclo[3.2.1]oct-3-yl)-2,2-diphenyl-propyl]-acetamide;
N-[3-((Endo)-8-methyl-8-aza-bicyclo[3.2.1]oct-3-yl)-2,2-diphenyl-propyl]-benzamide;
3-((Endo)-8-methyl-8-aza-bicyclo[3.2.1]oct-3-yl)-2,2-di-thiophen-2-yl-propionitrile;
(Endo)-3-(2-cyano-2,2-di-thiophen-2-yl-ethyl)-8,8-dimethyl-8-azonia-bicyclo[3.2.1]octane iodide;
N-[3-((Endo)-8-methyl-8-aza-bicyclo[3.2.1]oct-3-yl)-2,2-diphenyl-propyl]-benzenesulfonamide;
[3-((Endo)-8-methyl-8-aza-bicyclo[3.2.1]oct-3-yl)-2,2-diphenyl-propyl]-urea;
N-[3-((Endo)-8-methyl-8-aza-bicyclo[3.2.1]oct-3-yl)-2,2-diphenyl-propyl]-methanesulfonamide; and/or
(Endo)-3-{2,2-diphenyl-3-[(1-phenyl-methanoyl)-amino]-propyl}-8,8-dimethyl-8-azonia-bicyclo[3.2.1]octane bromide.

Particular compounds that be of use in the present invention include:
(Endo)-3-(2-methoxy-2,2-di-thiophen-2-yl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane bromide;
(Endo)-3-(2-carbamoyl-2,2-diphenyl-ethyl)-8,8-dimethyl-8-azonia-bicyclo[3.2.1]octane iodide;
(Endo)-3-(2-cyano-2,2-di-thiophen-2-yl-ethyl)-8,8-dimethyl-8-azonia-bicyclo[3.2.1]octane iodide; and/or
(Endo)-3-{2,2-diphenyl-3-[(1-phenyl-methanoyl)-amino]-propyl}-8,8-dimethyl-8-azonia-bicyclo[3.2.1]octane bromide.

In one embodiment, the combination comprises one or two other therapeutic agents in addition to the compound of the invention.

The invention thus provides, in a further aspect, a combination comprising a compound of formula (I) or a pharmaceutically acceptable derivative thereof such as a salt, together with a PDE4 inhibitor.

The invention thus provides, in a further aspect, a combination comprising a compound of formula (I) or a pharmaceutically acceptable derivative thereof such as a salt, together with a β₂-adrenoreceptor agonist.

The invention thus provides, in a further aspect, a combination comprising a compound of formula (I) or a pharmaceutically acceptable derivative thereof such as a salt, together with an anticholinergic.

The invention thus provides, in a further aspect, a combination comprising a compound of formula (I) or a pharmaceutically acceptable derivative thereof such as a salt, together with a H1 receptor antagonist.

The invention thus provides, in a further aspect, a combination comprising a compound of formula (I) or a pharmaceutically acceptable derivative thereof such as a salt, together with a corticosteroid.

The invention thus provides, in a further aspect, a combination comprising a compound of formula (I) or a pharmaceutically acceptable derivative thereof such as a salt, together with a A2a receptor agonist.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The individual compounds of such combinations may be administered either sequentially or simultaneously in separate or combined pharmaceutical formulations. Suitably, the individual compounds will be administered simultaneously in a combined pharmaceutical formulation. Appropriate doses of known therapeutic agents will be readily appreciated by those skilled in the art.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

The following Descriptions and Examples illustrate the preparation of compounds of the invention. The Examples are not to be considered as limiting the scope of the invention in any way.

### DETAILED DESCRIPTION

Throughout the examples, the following abbreviations are used:
LCMS: Liquid Chromatography Mass Spectrometry
RT: retention time
DMF: N,N-dimethylformamide
h: hour(s)
min: minute(s)
TBTU: O-(benzotriazol-1-yl)-N,N,N′,N′-tetramethyluronium tetrafluoroborate
PS-DIEA: polymer supported diisopropylethylamine

SCX cartridges are Ion Exchange SPE columns where the stationary phase is polymeric benzene sulfonic acid. These are used to isolate amines. SCX2 cartridges are Ion Exchange SPE columns where the stationary phase is polymeric propylsulfonic acid. These are used to isolate amines.

Organic solutions were dried either over magnesium or sodium sulfate.

LCMS was conducted on a Supelcosil LCABZ+PLUS column (3.3 cm×4.6 mm ID) eluting with 0.1% HCO₂H and 0.01M ammonium acetate in water (solvent A) and 0.05% HCO₂H 5% water in acetonitrile (solvent B), using the following elution gradient 0.0-7 min 0% B, 0.7-4.2 min 100% B, 4.2-5.3 min 0% B, 5.3-5.5min 0% B at a flow rate of 3 ml/min. The mass spectra were recorded on a Fisons VG Platform spectrometer using electrospray positive and negative mode (ES+ve and ES−ve).

The Flashmaster II is an automated multi-user flash chromatography system, available from Argonaut Technologies Ltd, which utilises disposable, normal phase, SPE cartridges (2 g to 100 g). It provides quaternary on-line solvent mixing to enable gradient methods to be run. Samples are queued using the multi-functional open access software, which manages solvents, flow-rates, gradient profile and collection conditions. The system is equipped with a Knauer variable wavelength uv-detector and two Gilson FC204 fraction-collectors enabling automated peak cutting, collection and tracking.

### Description 1

### 1,1-Dimethylethyl 4-[(4-bromophenyl)oxy]-1-piperidinecarboxylate

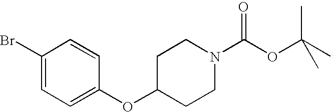

To a stirring solution of 4-bromophenol (5.19 g), 1,1-dimethylethyl 4-hydroxy-1-piperidinecarboxylate (Aldrich) (5.032 g) and triphenylphosphine (7.869 g) in dichloromethane (125 ml) was added, portionwise, di-t-butyl azodicarboxylate (6.908 g). The reaction mixture was stirred at room temperature under a nitrogen atmosphere for about 65 h. The solvent was evaporated from the mixture giving a viscous residue. A 3 g sample of this residue was purified by Flashmaster II on a 100 g silica cartridge using a 0-100% ethyl acetate in cyclohexane gradient over 80 minutes. The solvent was evaporated from appropriate fractions to give the title compound (604 mg). LCMS RT=3.72 min, ES+ve m/z 341/343 (M+H)⁺.

The remaining crude material was purified by flash column chromatography on Merck Kieselgel 60 (9385) silica (400 g) using 10% ethyl acetate in cyclohexane as the eluent. Evaporation of the solvent from appropriate fractions gave a colourless oil that was further purified by Flashmaster II on a 100 g silica cartridge using a 0-50% ethyl acetate in cyclohexane gradient over 60 minutes. Evaporation of the solvent from approriate fractions gave the title compound as a colourless oil that crystallised on standing (5.725 g). LCMS RT=3.72 mins, ES+ve m/z 341/343 (M+H)⁺.

### Description 2

### Phenylmethyl 4-{4-[(1-{[(1,1-dimethylethyl)oxy]carbonyl}-4-piperidinyl)oxy]phenyl}-4-hydroxy-1-piperidinecarboxylate

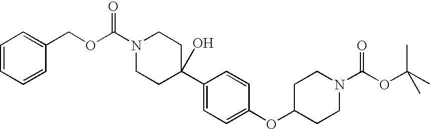

n-Butyl lithium (13.9 ml of 1.6M solution in hexane) was added dropwise to a stirring solution of 1,1-dimethylethyl 4-[(4-bromophenyl)oxy]-1-piperidinecarboxylate (D1) (6.32 g) in anhydrous tetrahydrofuran (75 ml) such that the reaction mixture temperature did not exceed −70° C. The resulting mixture was stirred at −75° C. for 20 min. A solution of phenylmethyl 4-oxo-1-piperidinecarboxylate (Aldrich) (5.173 g) in anhydrous tetrahydrofuran (15 ml) was then added dropwise such that the reaction temperature was maintained below −70° C. The reaction mixture was then allowed to warm slowly to room temperature overnight. The reaction mixture was partitioned between saturated aqueous ammonium chloride (200 ml) and dichloromethane (150 ml). The organic layer was washed with brine (100 ml), dried over anhydrous magnesium sulphate and evaporated to give a viscous residue. This material was purified by Flashmaster II on a 100 g silica cartridge eluted with a 0-50% ethyl acetate in cyclohexane gradient over 60 min. The solvent was evaporated from appropriate fractions to give the title compound (1.606 g). LCMS RT=3.59 min, ES+ve m/z 528 (M+NH₄)⁺.

Additional fractions containing the desired material were combined and evaporated to give a residue that was repurified by Flashmaster II using a 0-50% ethyl acetate in cyclohexane gradient. Evaporation of the solvent from approriate fractions gave the title compound as a colourless gum (521 mg). LCMS RT=3.59 mins, ES+ve m/z 528 (M+NH4)+.

### Description 3

### Phenylmethyl 4-[4-(4-piperidinyloxy)phenyl]-1-piperidinecarboxylate

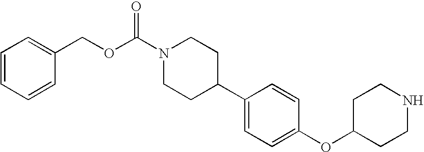

A stirring solution of phenylmethyl 4-{4-[(1-{[(1,1-dimethylethyl)oxy]carbonyl}-4-piperidinyl)oxy]phenyl}-4-hydroxy-1-piperidinecarboxylate (D2) (1.62 g) and triethylsilane (2.54 g) in anhydrous dichloromethane at −78° C. was treated, dropwise, with trifluoroacetic acid (1.18 ml). The resulting mixture was stirred at −78° C. for 15 min and at room temperature overnight. Evaporation of the solvent gave a residue that was re-dissolved in toluene (10 ml) and evaporated once more to give a semi-solid. This material was dissolved in methanol and applied to two 20 g SCX ion-exchange cartridges. The cartridges were eluted with methanol, followed by 2N ammonia in methanol. Evaporation of the solvent from the ammonia-containing fractions gave the title compound (945 mg). LCMS RT=2.59 min, ES+ve m/z 395 (M+H)⁺.

### Description 4

### Phenylmethyl 4-(4-{[1-(1-methylethyl)-4-piperidinyl]oxy}phenyl)-1-piperidinecarboxylate

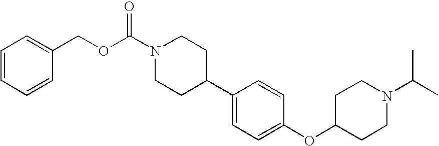

A stirring solution of phenylmethyl 4-[4-(4-piperidinyloxy)phenyl]-1-piperidinecarboxylate (D3) (614 mg), acetic acid (0.1 ml) and acetone (1 ml) in dichloromethane (10 ml) was treated with sodium triacetoxyborohydride (661 mg). The resulting reaction mixture was stirred at room temperature under a nitrogen atmosphere for approximately 20 h. Further quantities of acetone (1 ml) and sodium triacetoxyborohydride (500 mg) were added and the mixture stirred for an additional 4 h. The reaction mixture was partitioned between dichloromethane (50 ml) and saturated sodium bicarbonate solution (50 ml). The organic layer was washed with brine (50 ml), dried over anhydrous magnesium sulphate and evaporated to give the title compound (667 mg). LCMS RT=2.70 min, ES+ve m/z 437 (M+H)⁺.

### Description 5

### 1-(1-Methylethyl)-4-{[4-(4-piperidinyl)phenyl]oxy}piperidine

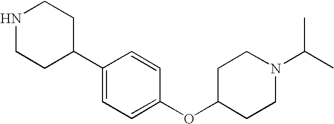

A solution of phenylmethyl 4-(4-{[1-(1-methylethyl)-4-piperidinyl]oxy}phenyl)-1-piperidinecarboxylate (D4) (667 mg) in ethanol (20 ml) was hydrogenated at atmospheric pressure in the presence of 10% wt palladium on carbon (300 mg). After 3 h the reaction mixture was filtered through celite and the filtrate evaporated. The residue was dissolved in methanol and applied to a 10 g SCX ion-exchange cartridge. The cartridge was eluted with methanol, followed by 2N ammonia in methanol. Evaporation of the solvent from the ammonia-containing fractions gave the title compound (416 mg). LCMS RT=1.5 min, ES+ve m/z 303 (M+H)⁺.

### Description 6

### Methyl 4-(azetidin-1-ylcarbonyl)benzoate

Methyl 4-chlorocarbonylbenzoate (2.0 g) was stirred with triethylamine (2.0 ml) in dichloromethane (20 ml) at 20° C. and azetidine (0.81 ml) was added. (It is to be noted that this reaction was vigorous, and thus cooling and slower addition of azetidine may be advisable.) After stirring at room temperature under nitrogen for 2 h, the solution was diluted with dichloromethane and washed with water twice. The organic solution was then washed with dilute hydrochloric acid (2N) and water, each time back extracting with dichloromethane. The combined organic layers were dried with magnesium sulphate and evaporated to give the title compound (1.92 g), LCMS RT=2.33 min. ES+ve m/z 220 (M+H)⁺.

### Description 7

### 4-(Azetidin-1-ylcarbonyl)benzoic Acid

Methyl 4-(azetidin-1-ylcarbonyl)benzoate (D6) (1.91g) was heated at 70° C. in MeOH (20 ml) and 2M sodium hydroxide solution (10 ml) with stirring for 1.5 h. After cooling, the mixture was acidified with 2M hydrochloric acid (10 mI) to pH 5. The white solid, which separated, was collected by filtration and washed with water. The combined filtrate and washings were acidified by adding further 2M hydrochloric acid to pH 1 and the solution was extracted three times with ethylacetate. The combined organic extracts were washed with brine, dried with magnesium sulphate and evaporated to give the title compound (0.25g). LCMS RT=2.04 min. ES+ve m/z 206 (M+H)⁺.

### EXAMPLE 1

### 1-{[4-(1-Azetidinylcarbonyl)phenyl]carbonyl}-4-(4-{[1-(1-methylethyl)-4-piperidinyl]oxy}phenyl)piperidine

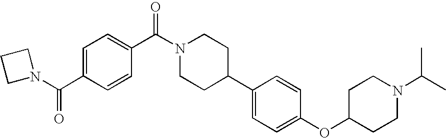

A solution of 1-(1-methylethyl)4-{[4-(4-piperidinyl)phenyl]oxy}piperidine (D5) (24.2 mg) in dichloromethane (1.5 ml) was added to a mixture of 4-(1-azetidinylcarbonyl)benzoic acid (D7) (17.4 mg), TBTU (28.9 mg) and PS-DIEA (100 mg of 3.88 mmol/g). The resulting mixture was agitated at room temperature in a sealed vial for about 65 h. The PS-DIEA was removed by filtration and washed with dichloromethane (3 ml). The combined filtrate and washings were evaporated under a stream of nitrogen giving a residue that was dissolved in methanol (3 ml) and applied to an SCX ion-exchange cartridge (1 g). The cartridge was eluted with methanol (3×3 ml) and then 2N ammonia in methanol (2×3 ml). Evaporation of the solvent from the ammonia-containing fractions gave the title compound (38.6 mg). LCMS RT=2.24 min, ES+ve m/z 490 (M+H)⁺.

Biological Data

Compound of the invention may be tested for in vitro biological activity in accordance with the following or similar assays:

H1 receptor cell line generation and FLIPR assay protocol

1. Generation of Histamine H1 Cell Line

The human H1 receptor was cloned using known procedures described in the literature [Biochem. Biophys. Res. Commun. 1994, 201(2), 894]. Chinese hamster ovary cells stably expressing the human H1 receptor were generated according to known procedures described in the literature [Br. J. Pharmacol. 1996, 117(6), 1071].

Histamine H1 Functional Antagonist Assay

The histamine H1 cell line was seeded into non-coated black-walled clear bottom 384-well tissue culture plates in alpha minimum essential medium (Gibco/Invitrogen, cat no. 22561-021), supplemented with 10% dialysed foetal calf serum (Gibco/Invitrogen cat no. 12480-021) and 2 mM L-glutamine (Gibco/Invitrogen cat no 25030-024) and maintained overnight at 5% CO₂, 37° C.

Excess medium was removed from each well to leave 10 μl. 30 μl loading dye (250 μM Brilliant Black, 2 μM Fluo-4 diluted in Tyrodes buffer+probenecid (145 mM NaCl, 2.5 mM KCl, 10 mM HEPES, 10 mM D-glucose, 1.2 mM MgCl₂, 1.5 mM CaCl₂, 2.5 mM probenecid, pH adjusted to 7.40 with NaOH 1.0 M)) was added to each well and the plates were incubated for 60 minutes at 5% CO₂, 37° C.

10 μl of test compound, diluted to the required concentration in Tyrodes buffer+probenecid (or 10 μl Tyrodes buffer+probenecid as a control) was added to each well and the plate incubated for 30 min at 37° C., 5% CO₂. The plates were then placed into a FLIPR™ (Molecular Devices, UK) to monitor cell fluorescence (λₑₓ=488 nm, λ_{EM}=540 nm) in the manner described in Sullivan et al. (In: Lambert DG (ed.), Calcium Signaling Protocols, New Jersey: Humana Press, 1999, 125-136) before and after the addition of 10 μl histamine at a concentration that results in the final assay concentration of histamine being EC₈₀.

Functional antagonism is indicated by a suppression of histamine induced increase in fluorescence, as measured by the FLIPRTM system (Molecular Devices). By means of concentration effect curves, functional affinities are determined using standard pharmacological mathematical analysis.

2. H3 Receptor Cell Line Generation, Membrane Preparation and Functional GTPγS Assay Protocols

Generation of Histamine H3 Cell Line

The histamine H3 cDNA was isolated from its holding vector, pCDNA3.1 TOPO (InVitrogen), by restriction digestion of plasmid DNA with the enzymes BamH1 and Not-1 and ligated into the inducible expression vector pGene (InVitrogen) digested with the same enzymes. The GeneSwitch™ system (a system where in transgene expression is switched off in the absence of an inducer and switched on in the presence of an inducer) was performed as described in U.S. Pat. Nos: 5,364,791; 5,874,534; and 5,935,934. Ligated DNA was transformed into competent DH5α E. coli host bacterial cells and plated onto Luria Broth (LB) agar containing Zeocin™ (an antibiotic which allows the selection of cells expressing the sh ble gene which is present on pGene and pSwitch) at 50 μg ml⁻¹. Colonies containing the re-ligated plasmid were identified by restriction analysis. DNA for transfection into mammalian cells was prepared from 250 ml cultures of the host bacterium containing the pGeneH3 plasmid and isolated using a DNA preparation kit (Qiagen Midi-Prep) as per manufacturers guidelines (Qiagen).

CHO K1 cells previously transfected with the pSwitch regulatory plasmid (InVitrogen) were seeded at 2×10e6 cells per T75 flask in Complete Medium, containing Hams F12 (GIBCOBRL, Life Technologies) medium supplemented with 10% v/v dialysed foetal bovine serum, L-glutamine, and hygromycin (100 μg ml⁻¹), 24 hours prior to use. Plasmid DNA was transfected into the cells using Lipofectamine plus according to the manufacturers guidelines (InVitrogen). 48 hours post transfection cells were placed into complete medium supplemented with 500 μg ml⁻¹ Zeocin™.

10-14 days post selection 10 nM Mifepristone (InVitrogen), was added to the culture medium to induce the expression of the receptor. 18 hours post induction cells were detached from the flask using ethylenediamine tetra-acetic acid (EDTA; 1:5000; InVitrogen), following several washes with phosphate buffered saline pH 7.4 and resuspended in Sorting Medium containing Minimum Essential Medium (MEM), without phenol red, and supplemented with Earles salts and 3% Foetal Clone II (Hyclone). Approximately 1×10e7 cells were examined for receptor expression by staining with a rabbit polyclonal antibody, 4a, raised against the N-terminal domain of the histamine H3 receptor, incubated on ice for 60 minutes, followed by two washes in sorting medium. Receptor bound antibody was detected by incubation of the cells for 60 minutes on ice with a goat anti rabbit antibody, conjugated with Alexa 488 fluorescence marker (Molecular Probes). Following two further washes with Sorting Medium, cells were filtered through a 50 μm Filcon™ (BD Biosciences) and then analysed on a FACS Vantage SE Flow Cytometer fitted with an Automatic Cell Deposition Unit. Control cells were non-induced cells treated in a similar manner. Positively stained cells were sorted as single cells into 96-well plates, containing Complete Medium containing 500 μg ml⁻¹ Zeocin™ and allowed to expand before reanalysis for receptor expression via antibody and ligand binding studies. One clone, 3H3, was selected for membrane preparation.

Membrane Preparation from Cultured Cells

All steps of the protocol are carried out at 4° C. and with pre-cooled reagents. The cell pellet is resuspended in 10 volumes of homogenisation buffer (50 mM N-2-hydroxyethylpiperazine-N′-2-ethanesulfonic acid (HEPES), 1 mM ethylenediamine tetra-acetic acid (EDTA), pH 7.4 with KOH, supplemented with 10e-6M leupeptin (acetyl-leucyl-leucyl-arginal; Sigma L2884), 25 μg/ml bacitracin (Sigma B0125), , 1 mM phenylmethylsulfonyl fluoride (PMSF) and 2×10e-6M pepstain A (Sigma)). The cells are then homogenised by 2×15 second bursts in a 1 litre glass Waring blender, followed by centrifugation at 500 g for 20 minutes. The supernatant is then spun at 48,000 g for 30 minutes. The pellet is resuspended in homogenisation buffer (4× the volume of the original cell pellet) by vortexing for 5 seconds, followed by homogenisation in a Dounce homogeniser (10-15 strokes). At this point the preparation is aliquoted into polypropylene tubes and stored at −80° C.

Histamine H3 Functional Antagonist Assay

For each compound being assayed, in a solid white 384 well plate, is added:-
(a) 0.5 μl of test compound diluted to the required concentration in DMSO (or 0.5 μl DMSO as a control);
(b) 30 μl bead/membrane/GDP mix prepared by mixing Wheat Germ Agglutinin Polystyrene LeadSeeker® (WGA PS LS) scintillation proximity assay (SPA) beads with membrane (prepared in accordance with the methodology described above) and diluting in assay buffer (20 mM N-2-Hydroxyethylpiperazine-N′-2-ethanesulfonic acid (HEPES)+100 mM NaCl+1 mM MgCl₂, pH7.4 NaOH) to give a final volume of 30 μl which contains 5 μg protein and 0.25 mg bead per well, incubating at room temperature for 60 minutes on a roller and, just prior to addition to the plate, adding 10 μM final concentration of guanosine 5′ diphosphate (GDP) (Sigma; diluted in assay buffer);
(c) 15 μl 0.38 nM [³⁵S]-GTPγS (Amersham; Radioactivity concentration=37MBq/ml; Specific activity=1160Ci/mmol), histamine (at a concentration that results in the final assay concentration of histamine being EC₈₀).

After 2-6 hours, the plate is centrifuged for 5 min at 1500 rpm and counted on a Viewlux counter using a 613/55 filter for 5 min/plate. Data is analysed using a 4-parameter logistical equation. Basal activity used as minimum i.e. histamine not added to well.

CNS Penetration

Compounds are dosed intravenously at a nominal dose level of 1 mg/kg to male CD Sprague Dawley rats. Compounds are formulated in 5% DMSO/45% PEG200/50% water. Blood samples are taken under terminal anaesthesia with isoflurane at 5 minutes post-dose and the brains are also removed for assessment of brain penetration. Blood samples are taken directly into heparinised tubes. Blood samples are prepared for analysis using protein precipitation and brain samples are prepared using extraction of drug from brain by homogenisation and subsequent protein precipitation. The concentration of parent drug in blood and brain extracts is determined by quantitative LC-MS/MS analysis using compound-specific mass transitions.

Rat Pharmacokinetics

Compounds are dosed to male CD Sprague Dawley rats by single intravenous or oral administration at a nominal dose level of 1 mg/kg and 3 mg/kg respectively. Compounds are formulated in 5% DMSO/45% PEG200/50% water. An intravenous profile is obtained by taking serial or terminal blood samples at 0.083, 0.25, 0.5, 1, 2, 4, and 7 hours post dose. An oral profile is obtained by taking serial or terminal blood samples at 0.25, 0.5, 1, 2, 4, 7 and 12 hours post dose. Blood samples are taken directly into heparinised tubes. Blood samples are prepared by protein precipitation and subjected to quantitative analysis by LC-MS/MS using compound-specific mass transitions. Drug concentration-time profiles are generated and non-compartmental PK analysis used to generate estimates of half-life, clearance, volume of distribution and oral bioavailability.

Dog Pharmacokinetics

Compounds are dosed to male Beagle dogs by single intravenous or oal administration at a nominal dose level of 1 mg/kg and 2 mg/kg respectively. The study is carried out according to a crossover design such that the same dog is used for both dosing events and the dosing events occurred 1 week apart. Compounds are formulated in 5% DMSO/45% Peg200/50% water. An intravenous profile is obtained by taking serial blood samples at 0.083, 0.25, 0.5, 0.75, 1, 2, 4, 6 & 12 hr post dose. An oral profile is obtained by taking serial blood samples at 0.25, 0.5, 0.75, 1, 2, 4, 6, 12 & 24 hr post dose. Blood samples are taken directly into heparinised tubes. Blood samples are prepared by protein precipitation and subjected to quantitative analysis by LC-MS/MS using compound-specific mass transitions. Drug concentration-time profiles are generated and non-compartmental PK analysis used to generate estimates of half-life, clearance, volume of distribution and oral bioavailability.

Results

In the above assays or similar assays compound of formula (I) had
(i) an average pki (pkb) at H3 of approximately 9.6
(ii) an average pki (pkb) at H1 of approximately 5.6
(iii) low CNS penetration (less than 100 ng compound/g of brain tissue)
(iv) good oral bioavailability (in the rat about 22% F and in the dog about 87% F)
(v) half-life in the rat of greater than about 2 hrs and in the dog greater than about 4 hours

The content of all documents including literature references, patents and patent applications referred to hereinabove are to be considered as incorporated in full herein.

We claim:

1. 1-{[4-(1-Azetidinylcarbonyl)phenyl]carbonyl}4-(4-{[1-(1-methylethyl)4-piperidinyl]oxy}phenyl)piperidine 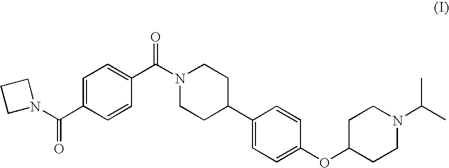
and derivatives thereof.

2. Compound according to claim 1 wherein the derivative is a salt or solvate.

3. Compound according to claim 1 wherein the derivative is a pharmaceutically acceptable salt or solvate.

4. A process for the preparation of a compound of formula (I) or a derivative thereof, the process comprising reacting a compound of Formula (III) 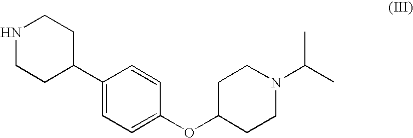
or a derivative thereof, with 4-(azetidin-1-ylcarbonyl) benzoic acid.

5. A pharmaceutical composition which comprises a compound or a pharmaceutically acceptable derivative thereof according to claim 1 optionally with one or more pharmaceutically acceptable carriers and/or excipients.

6. A pharmaceutical composition according to claim 5 which further comprises an H1 receptor antagonist.

7. A pharmaceutical combination comprising a compound or a pharmaceutically acceptable derivative thereof according to claim 1 and a H1 receptor antagonist.

8. A method for the treatment or prophylaxis of inflammatory and/or allergic disorders which comprises administering to a patient in need thereof an effective amount of a compound or a pharmaceutically acceptable derivative thereof according to claim 1.
